(19) United States
(12) Patent Application Publication
(10) Pub. No.: US 2024/0336388 A1
(43) Pub. Date: Oct. 10, 2024

(54) SYSTEM FOR PROCESSING SUCTION BAGS

(71) Applicant: DEF'HYGIE, AMBERIEUX d'AZERGUES (FR)

(72) Inventors: Benoit DUMONCEAU, LACHASSAGNE (FR); Raphaël POYARD, CHESSY (FR); Laurent MAIROT, MONTPELLIER (FR)

(21) Appl. No.: 18/697,217

(22) PCT Filed: Oct. 3, 2022
(86) PCT No.: PCT/FR2022/051867

§ 371 Date: Mar. 29, 2024

(30) Foreign Application Priority Data
Oct. 1, 2021 (FR) FR21/10410

(51) Int. Cl. B65B 69/00; A61M 1/00; B65F 1/14; C02F 1/50; C02F 103/00

(52) U.S. Cl. CPC B65B 69/0016; A61M 1/69; B65F 1/1426; C02F 1/50; C02F 2103/003

(57) ABSTRACT

A system for processing suction bags including a housing comprising a loading area configured to allow suction bags to be loaded into the housing, a cutting area, a processing area, and a storage area. It also comprises a rotary support which comprises multiple receiving sites configured to accommodate suction bags, and which is mounted so as to be rotatable about a first axis of rotation; and a cutting member which is located in the cutting area and which is configured to cut at least one suction bag received in a receiving site when the at least one suction bag is located in the cutting area.

### CROSS REFERENCE TO RELATED APPLICATIONS

This application is a National Stage of PCT Application No. PCT/FR2022/051867 filed on Oct. 3, 2022, which claims priority to French Patent Application No. 21/10410 filed on Oct. 1, 2021, the contents each of which are incorporated herein by reference thereto.

### TECHNICAL FIELD

The present disclosure relates to a processing system, intended to open and process suction bags that could contain liquids and/or body fluids.

The present disclosure finds a favorite, yet non-limiting, application in the processing of suction bags that could be used in the medical and hospital field in order to recover body fluids originating from patients. Applications could be considered in the industrial field for example in order to separate and process bags and their contents.

Indeed, the automated separation of a fluid initially located in a pouch allows limiting contact between a user and the fluid contained in the pouch, which is advantageous in particular when the fluids might present any risks to the user.

### BACKGROUND

Thus, it is known from the prior art and from the document CN212419000U to use a processing system including a cutting element mounted movable in rotation about a vertical axis so as to define a horizontal cutting plane, the cutting element being configured to open in the widthwise direction, suction bags positioned beforehand in a main housing.

This type of processing system allows performing opening of suction bags easily and without contact between a user and the content of the suction bags. However, such a processing system has major drawbacks, the first one being the impossibility of adding one or several suction bag(s) in the main housing as long as the processing cycle of the suction bags already in place is not completed. Moreover, opening of suction bags in the widthwise direction thereof is not optimum, since part of the content of the suction bag might remain in the lower portion of the suction bag after opening of the latter in the widthwise direction.

### BRIEF SUMMARY

Hence, the technical problem at the origin of the present disclosure consists in providing a system for processing suction bags allowing ensuring an improved emptying of the suction bags and enabling the insertion of suction bags inside the processing system simultaneously with the processing of bags already present in the processing system, while avoiding contact between a user and the content of the suction bags.

To this end, the present disclosure relates to a system for processing suction bags, and more particularly suction bags containing liquid wastes, and for example liquid medical wastes, comprising:
a housing including a loading area configured to enable loading of suction bags within the housing, a cutting area in which suction bags are intended to be cut, a processing area in which liquid wastes originating from cut suction bags are intended to be processed, and a storage area in which processed suction bags are intended to be stored,
a rotary support which is housed at least partially in the housing and which is mounted movable in rotation about a first axis of rotation, the rotary support comprising several receiving sites each configured to accommodate a suction bag, the rotary support being configured to displace suction bags received in the receiving sites from the loading area towards the cutting area and then towards the storage area,
a cutting element which is located in the cutting area and which is mounted movable in rotation about a second axis of rotation which is substantially horizontal, the cutting element defining a cutting plane which is substantially vertical and being configured to cut at least one suction bag received in a receiving site and located in the cutting area.

Thus, the processing system according to the present disclosure allows performing opening of the suction bags easily and without contact between a user and the contents of the suction bags, while allowing adding one or several suction bag(s) in the housing simultaneously with the processing of at least one suction bag located in the cutting area and/or with the discharge of a suction bag in the storage area.

Moreover, opening suction bags in their lengthwise direction allows optimally recovering the contents of such a suction bag.

In the present text, by “liquid medical wastes”, it should be understood, in particular, liquid wastes with infectious risks (DASRI) and/or with chemical risks originating, for example, from different healthcare and medical-social facilities, from the industry, as well as body fluids.

The processing system may further have one or more of the following features, which may be considered alone or in combination.

According to one embodiment, the first axis of rotation is substantially vertical.

According to one embodiment, the cutting plane is tangent to a virtual circle, the center of which is located on the first axis of rotation of the rotary support. In other words, the second axis of rotation of the cutting element extends radially with respect to the first axis of rotation of the rotary support.

According to one embodiment, the cutting element is movably mounted relative to the housing according to a displacement direction which is substantially vertical. Thus, the cutting element allows optimally cutting suction bags of different sizes.

According to one embodiment, the loading area of the processing system includes at least one upper insertion opening configured to enable simultaneous positioning of a plurality of suction bags in receiving sites of the rotary support located opposite the at least one upper insertion opening.

According to one embodiment, the receiving sites are angularly offset relative to one another with respect to the first axis of rotation.

According to one embodiment, the receiving sites are located at a same distance from the first axis of rotation.

According to one embodiment, the processing system includes between two and ten receiving sites, and for example six receiving sites.

According to one embodiment, the processing system further includes a drive mechanism configured to drive the rotary support in rotation about the first axis of rotation.

According to one embodiment, the drive mechanism includes an electric actuator, such as an electric motor, a pneumatic actuator, or a manual drive mechanism for example provided with an actuating lever.

According to one embodiment, the processing system includes a plurality of holding devices, each holding device being associated with a respective receiving site and being configured to hold in position a suction bag received in the respective receiving site during cutting of said suction bag by the cutting element.

According to one embodiment, each device for holding the processing system includes at least one passage opening which is substantially vertical and which is configured to enable the passage of the cutting element.

According to one embodiment, each holding device includes a plurality of holding studs. For example, each holding stud extends from a lower face of the rotary support, and extends substantially vertically in the direction of the ground.

According to one embodiment, each receiving site includes an upper support surface which extends substantially horizontally and on which a suction bag received in said receiving site is intended to rest at least partially.

According to one embodiment, the housing includes a support portion which is located substantially in the same plane as the upper support surfaces and which is configured to at least partially support the suction bags during displacements thereof in the cutting area. Thus, when a suction bag is displaced in the cutting area, said suction bag is supported in part by the upper support surface of the respective receiving site and in part by the support portion, which allows holding said suction bag substantially vertically during displacement thereof in the cutting area.

According to one embodiment, the processing system further comprises at least one device for processing liquid wastes, said liquid wastes processing device being disposed in the processing area and being configured to collect and process liquid wastes originating from suction bags cut in the cutting area.

According to one embodiment, the liquid wastes processing device includes a compartment configured to contain liquid wastes originating from the suction bags cut in the cutting area.

According to one embodiment, the liquid wastes processing device includes a biocidal agent source and is configured to inject biocidal agent originating from the biocidal agent source into the compartment. Advantageously, the liquid wastes processing device is configured to inject biocidal agent originating from the biocidal agent source into the compartment when the compartment contains a predetermined liquid level.

According to one embodiment, the liquid wastes processing device includes an additional compartment configured to contain liquids originating from the suction bags cut in the cutting area.

According to one embodiment, the liquid wastes processing device is configured to direct at least part of the liquid wastes, originating from cut suction bags, towards the additional compartment.

According to one embodiment, the processing system includes discharge means configured to discharge the liquid wastes contained in the compartment and/or the additional compartment respectively out of the compartment and/or the additional compartment. Advantageously, the discharge means are disposed in the housing.

According to one embodiment, the discharge means are configured to discharge the liquid wastes contained in the compartment and/or the additional compartment by gravity flow, by injection of compressed air respectively into the compartment and/or the additional compartment or by generation of a depression at the level of a discharge opening provided on the compartment or the additional compartment.

Such a configuration of the liquid wastes processing device allows ensuring a suitable contact time between the liquid wastes and the biocidal agent in the compartment and/or the additional compartment. Once the time of contact between the collected liquid wastes and the biocidal agent is reached, it is possible to proceed with the discharge of the processed liquid wastes out of the compartment and/or the additional compartment.

According to one embodiment, the processing system further comprises a storage device disposed in the storage area and configured to receive and store one or several processed suction bag(s), said storage device being at least partially open upwards.

According to one embodiment, the housing and the rotary support delimit a discharge opening located substantially vertically over the storage area, the discharge opening being configured to enable the fall by gravity of a suction bag, processed beforehand, in the storage device when the respective receiving site is located substantially vertically over the storage area.

According to another embodiment, the processing system includes one or several ejection means configured to eject a processed suction bag out of the rotary support when the respective receiving site is located opposite the storage area.

According to one embodiment, the processing system further comprises a cover movably mounted on an upper portion of the housing between an opening position in which the cover enables access to the rotary support and a closure position in which the cover prevents access to the rotary support. Advantageously, the cover is configured to at least partially cover the upper insertion opening when the cover occupies the closure position, and to at least partially clear the upper insertion opening when the cover occupies the opening position.

According to one embodiment, the cover is pivotably mounted about a pivot axis which is substantially horizontal.

According to an embodiment of the present disclosure, the processing system, each of the receiving sites includes a gripping device configured to alternately occupy a holding position in which the gripping device hold in position the suction bag positioned in one of the receiving sites, and a release position configured to release the suction bag.

According to an embodiment of the present disclosure, the gripping device is configured to alternately occupy a holding position in which the suction bag rests on an upper face of the gripping device in order to hold the suction bag in position inside the respective receiving site, and a release position in which the suction bag cannot rest on the upper face of the gripping device.

According to an embodiment of the present disclosure, each of the gripping devices further includes a first clamp portion and a second clamp portion.

According to an embodiment of the present disclosure, the processing system further includes an actuation device configured to activate the release position of each of the gripping devices when the respective receiving site is positioned in a predetermined position.

According to an embodiment of the present disclosure, the actuation device may be a cam track for example.

According to an embodiment of the present disclosure, the predetermined position is located in the storage area.

### BRIEF DESCRIPTION OF THE FIGURES

The present disclosure will be well understood from the following description with reference to the appended figures, in which identical reference signs correspond to elements that are structurally and/or functionally identical or similar.

FIG. 1 is a perspective exploded view of a processing system according to the present disclosure;

FIG. 2 is a partial perspective top view of the processing system of FIG. 1;

FIG. 3 is a perspective top view of a rotary support and of a cutting area of the processing system of FIG. 1;

FIG. 4 is a perspective top view of the rotary support of FIG. 3;

FIG. 5 is a perspective side view of the rotary support of FIG. 3 and of a cutting element of the processing system of FIG. 1;

FIG. 6 is another perspective side view of the rotary support of FIG. 3 and of the cutting element of FIG. 5;

FIG. 7 is a perspective top view of the cutting area of the processing system of FIG. 1;

FIG. 8 is a perspective top view of the cutting area and of a liquid wastes processing device of the processing system of FIG. 1;

FIG. 9 is a bottom view of the rotary support of FIG. 3 and of the cutting element of FIG. 5;

FIG. 10 is another exploded perspective view of the processing system of FIG. 1;

FIG. 11 is a perspective view of the processing system according to a second embodiment of the present disclosure;

FIG. 12 is a perspective bottom view of the rotary support of the processing system of FIG. 11; and

FIG. 13 is a perspective top view of the actuation device used in the embodiment of FIG. 11.

### DETAILED DESCRIPTION

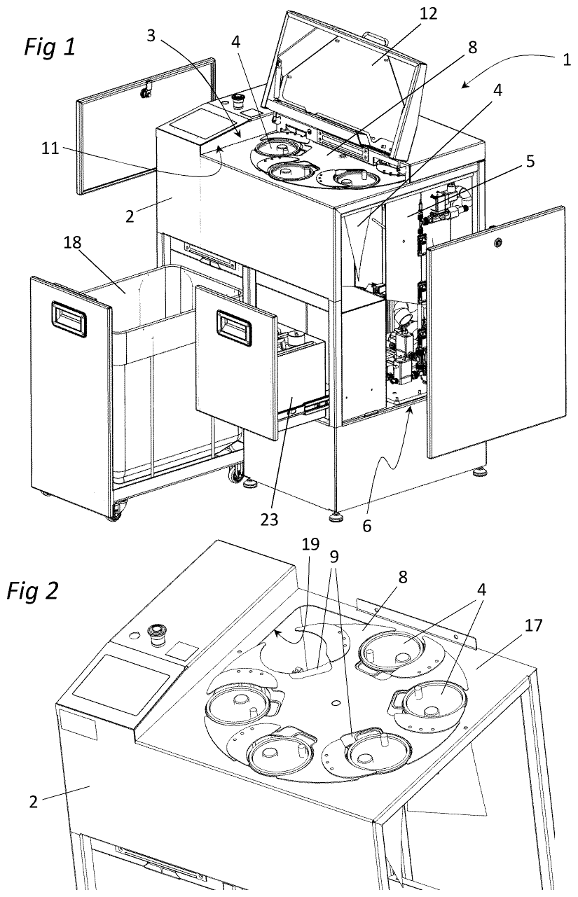

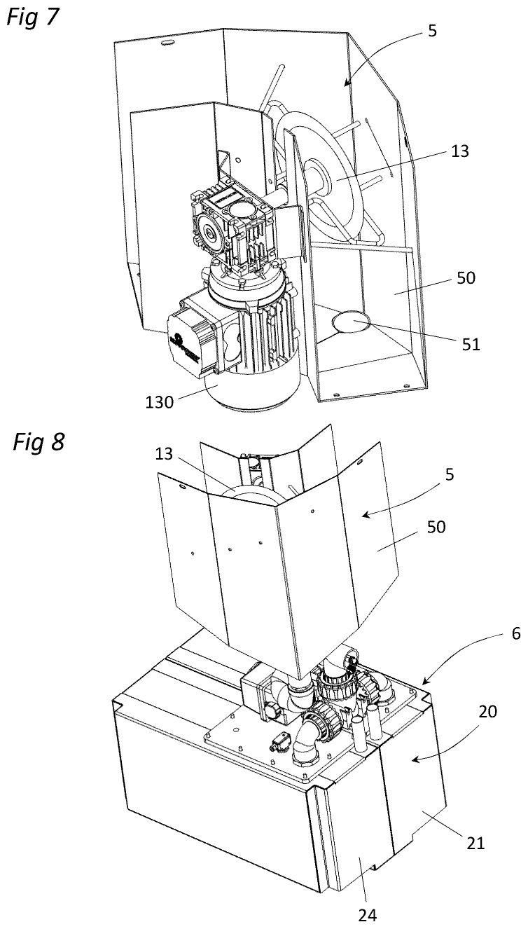

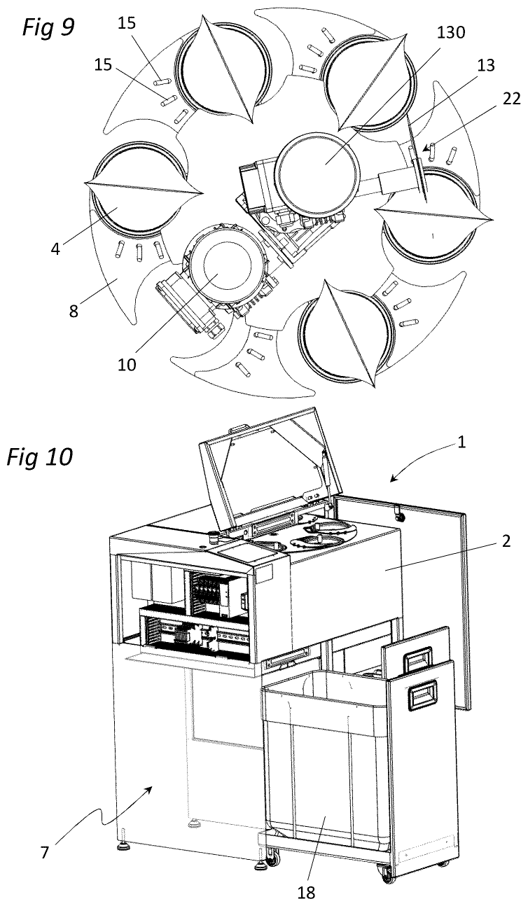

FIGS. 1 to 10 represent a processing system 1 configured to process suction bags 4, and more particularly suction bags 4 containing bodily fluids to be processed, such as blood, urine.

The processing system 1 comprises a housing 2 which may for example have a generally parallelepiped shape. In particular, the housing 2 includes a loading area 3 configured to enable loading of suction bags 4 within the housing 2. The housing 2 also includes a cutting area 5 in which suction bags 4 are intended to be cut, and a processing area 6 in which liquid wastes, originating from cut suction bags 4, are intended to be processed. The housing 2 further includes a storage area 7 in which the processed suction bags 4 are intended to be stored.

As shown more particularly in FIGS. 2 to 6, the processing system 1 further includes a rotary support 8 which is housed at least partially in the housing 2 and which is mounted movable in rotation about a first axis of rotation A1. According to the embodiment visible in the figures, the first axis of rotation A1 is substantially vertical.

The rotary support 8 comprises several receiving sites 9 each configured to accommodate a suction bag 4. The rotary support 8 is configured to displace suction bags 4 received in the receiving sites 9 from the loading area 3 towards the cutting area 5 and then towards the storage area 7.

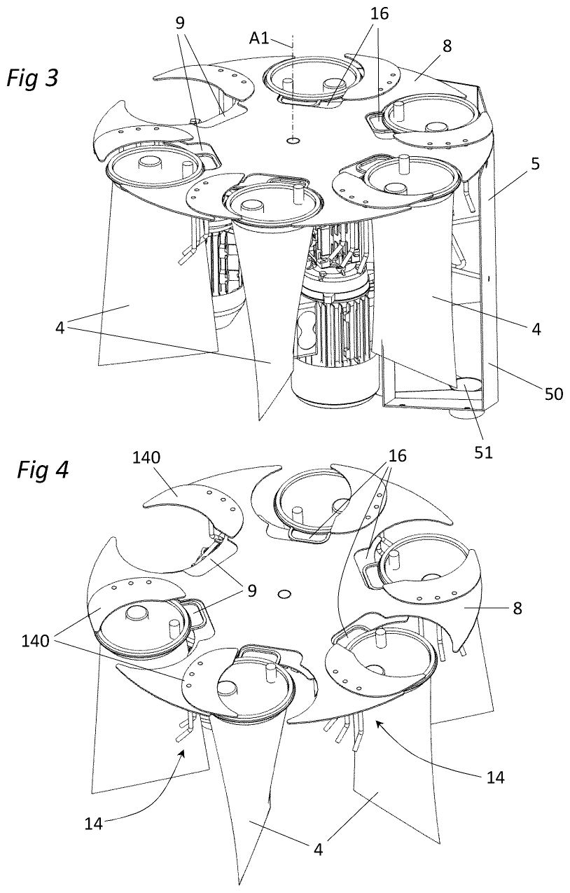

As shown more particularly in FIG. 3, the receiving sites 9 are distributed, and advantageously evenly distributed, around the first axis of rotation A1, and are located at a same distance from the first axis of rotation A1. The processing system 1 may include between two and ten receiving sites 9, and for example six receiving sites 9.

The rotation system 1 further includes a drive mechanism configured to drive the rotary support 8 in rotation about the first axis of rotation A1. For example, the drive mechanism may include an electric actuator, such as an electric motor 10, a pneumatic actuator, or a manual drive mechanism, such as an actuating lever.

According to the embodiment represented in the figures, the loading area 3 includes an upper insertion opening 11 configured to enable positioning of a plurality of suction bags 4 simultaneously in the receiving sites 9 of the rotary support 8 which are located opposite the upper insertion opening 11. However, the loading area 3 could include several upper insertion openings 11 each having dimensions corresponding substantially to those of the receiving sites 9, and thus enabling simultaneous positioning of a plurality of suction bags 4, for example three, in receiving sites 9 of the rotary support 8 which are respectively located opposite the upper insertion openings 11.

According to the embodiment represented in the figures, the processing system 1 further comprises a cover 12 movably mounted on an upper portion of the housing 2 between a closure position in which the cover 12 at least partially covers the upper insertion opening 11 and prevents access to the rotary support 8 and therefore to the receiving sites 9 located opposite the upper insertion opening 11, and an opening position in which the cover 12 at least partially clears the upper insertion opening 11 and enables access to the rotary support 8 and therefore to the receiving sites 9 located opposite the upper insertion opening 11. Advantageously, the cover 12 is pivotably mounted about a horizontal pivot axis which is located proximate to an upper face of the housing 2.

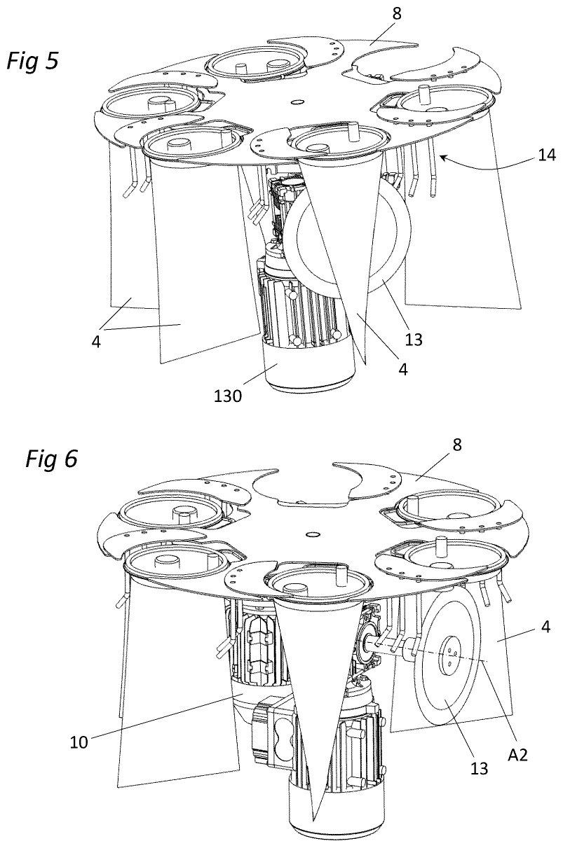

As shown more particularly in FIGS. 5 to 7, the processing system 1 also includes a cutting element 13 which is located in the cutting area 5 and which is mounted movable in rotation about a second axis of rotation A2 which is substantially horizontal. The cutting element 13 defines a cutting plane which is substantially vertical and which is configured to cut a suction bag 4 received in a receiving site 9 and located in the cutting area 5. Advantageously, the cutting plane defined by the cutting element 13 is tangent to a virtual circle, the center of which is located on the first axis of rotation A1 of the rotary support 8. Advantageously, the processing system 1 includes an electric drive motor 130 configured to drive the cutting element 13 in rotation.

The processing system 1 further includes a collector 50 defining the cutting area 5, and configured to collect the liquid wastes falling by gravity from the bags cut by the cutting element 13.

According to the embodiment represented in the figures, the processing system 1 also includes a plurality of holding devices 14. Each holding device 14 is associated with a respective receiving site 9, and is configured to hold in position a suction bag 4 received in the respective receiving site 9 when cutting said suction bag 4 by the cutting element 13. Advantageously, each holding device 14 includes a passage opening 22 which is substantially vertical and which is configured to enable the passage of the cutting element 13. According to the embodiment represented in the figures, each holding device 14 includes a plurality of holding studs 15, and for example three holding studs 15, configured to cooperate with a lateral wall of a suction bag received in the respective receiving site 9. Each holding stud 15 extends from a lower face of the rotary support 8, and extends substantially vertically in the direction of the ground.

According to the embodiment represented in the figures, each holding device 14 further includes a bearing member 140 configured to partially cover a suction bag 4 received in the respective receiving site 9, and more particularly to cooperate with an upper face of said suction bag 4 so as to form a vertical stop. Advantageously, the rotary support 8 and the housing 2 are configured to enable, after set-up of suction bags 4 in their respective receiving sites 9 and closure of the cover 12, a relative displacement between the rotary support 8 and said suction bags 4 during the first phase of rotation of the rotary support 8, and such that each of the suction bags 4 that have just been positioned on the rotary support 8 is displaced, relative to the rotary support 8, from a first position (cf. FIG. 3) in which the respective bearing member 140 is offset with respect to the respective suction bag at a second position (cf. FIG. 4) in which the respective bearing member 140 partially covers the respective suction bag.

As shown more particularly in FIG. 4, each receiving site 9 includes an upper support surface 16 which extends substantially horizontally and on which a suction bag 4 received in said receiving site 9 is intended to partially rest.

As shown in FIG. 2, the housing 2 includes a support portion 17 which is located substantially in the same plane as the upper support surfaces 16 and which is configured to partially support the suction bags 4 in particular during displacements thereof in the cutting area 5. Thus, when a suction bag 4 is displaced in the cutting area 5, said suction bag 4 is supported in part by the upper support surface 16 of the respective receiving site 9 and in part by the support portion 17, which allows holding said suction bag 4 substantially vertically during displacement thereof in the cutting area 5.

According to the embodiment represented in the figures, the processing system 1 further includes a storage device 18 which is disposed in the storage area 7 and which is configured to receive and store one or several processed suction bag(s) 4. Advantageously, the storage device 18 is at least partially open upwards. According to the embodiment represented in the figures, the housing 2 and the rotary support 8 delimit a discharge opening 19 which is located substantially vertically over the storage area 7, and more particularly vertically over the storage device 18, and which is configured to enable the fall by gravity of a suction bag 4, processed beforehand, in the storage device 18 when the respective receiving site 9 is located substantially vertically over the storage area 7.

According to another embodiment not represented in the figures, the processing system 1 could include one or several ejection means configured to eject a processed suction bag 4 out of the rotary support 8 when the respective receiving site 9 is located opposite the storage area 7.

According to the embodiment represented in the figures, the processing system 1 further comprises at least one liquid wastes processing device 20 which is disposed in the processing area 6 and which is configured to collect and process liquid wastes originating from suction bags 4 cut in the cutting area 5.

According to the embodiment represented in the figures, the liquid wastes processing device 20 includes a compartment 21 configured to contain liquid wastes originating from the suction bags 4 cut in the cutting area 5. The compartment 21 is configured to be fluidly connected to the collector 50, and more particularly to a lower opening 51 of said collector 50.

Advantageously, the liquid wastes processing device 20 includes a biocidal agent source 23 and is configured to inject biocidal agent originating from the biocidal agent source 23 into the compartment 21. More particularly, the liquid wastes processing device 20 is configured to inject biocidal agent originating from the biocidal agent source 23 into the compartment 21 when the compartment 21 contains a predetermined liquid level.

According to the embodiment not represented in the figures, the liquid wastes processing device 20 includes an additional compartment 24 also configured to contain liquids originating from the suction bags 4 cut in the cutting area 5. Advantageously, the liquid wastes processing device 20 is configured to direct at least part of the liquid wastes, originating from cut suction bags 4, towards the additional compartment 24 when the compartment 21 contains a predetermined liquid level. To this end, the liquid wastes processing device 20 may for example include a three-way valve and an actuator configured to displace the three-way valve between two different operating positions. The processing system 1 further includes discharge means (not represented in the figures) configured to discharge the processed liquid wastes contained in the compartment 21 and/or the additional compartment 24. Advantageously, the discharge means are disposed in the housing 2. For example, the discharge means may be configured to discharge the processed liquid wastes contained in the compartment 21 and/or the additional compartment 24 by gravity flow or by injection of compressed air respectively into the compartment 21 and/or the additional compartment 24.

Such a configuration of the liquid wastes processing device 20 allows ensuring a suitable contact time between the liquid wastes and the biocidal agent in the compartment 21 and/or the additional compartment 24. Once the time of contact between the collected liquid wastes and the biocidal agent is reached, it is possible to proceed with the discharge of the processed liquid wastes out of the compartment 21 and/or of the additional compartment 24.

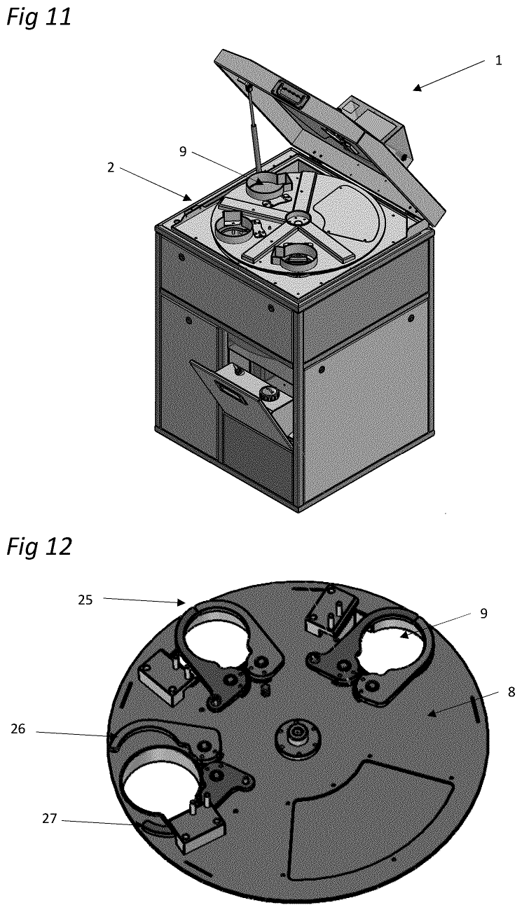

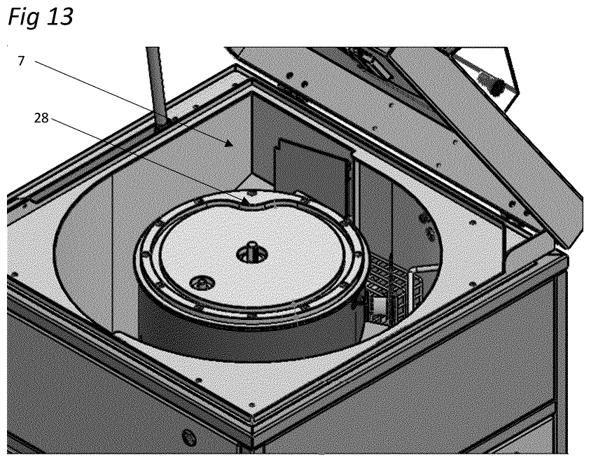

A second embodiment of the present disclosure is shown in FIGS. 11 to 13. The second embodiment of the present disclosure differs from the first embodiment in that each of the receiving sites 9 of the rotary support 8 includes a gripping device 25 configured to alternately occupy a holding position and a release position. Each of the gripping devices 25 includes a first clamp portion 26 and a second clamp portion 27. When the gripping device 25 is in the holding position, the suction bag 4, positioned in the respective receiving site 9, rests on an upper portion of the gripping device 25 formed by the first clamp portion 26 and the second clamp portion 27. Conversely, when the gripping device 25 is in the release position, the second clamp portion 27 is no longer in contact with the first clamp portion 26, which prevents the suction bag 4 from resting on the upper portion of the gripping device 25.

In this second embodiment of the present disclosure, the processing system 1 further includes an actuation device 28, such as a cam track for example, which is configured to activate and deactivate the release position of each of the gripping devices 25 when the respective receiving site 9 is positioned in a predetermined position. Advantageously, the predetermined position is provided in the storage area 7 and in line with the storage device 18 so that each of the suction bags 4 is released from the gripping device 25 in order to fall by gravity inside the storage device 18.

Of course, the present disclosure is in no way limited to the described and limited embodiment which has been given only as example. Modifications are still possible, in particular with regards to the construction of the various elements or by substitution with technical equivalents, yet without departing from the scope of the disclosure.

1. A system for processing suction bags, comprising:
a housing including a loading area configured to enable loading of suction bags within the housing, a cutting area in which suction bags are intended to be cut, a processing area in which liquid wastes, originating from cut suction bags, are intended to be processed, and a storage area in which processed suction bags are intended to be stored,
a rotary support which is housed at least partially in the housing and which is mounted movable in rotation about a first axis of rotation, the rotary support comprising several receiving sites each configured to accommodate a suction bag, the rotary support being configured to displace suction bags received in the receiving sites from the loading area towards the cutting area and then towards the storage area,
a cutting element which is located in the cutting area and which is mounted movable in rotation about a second axis of rotation which is substantially horizontal, the cutting element defining a cutting plane which is substantially vertical and being configured to cut at least one suction bag received in a receiving site and located in the cutting area.

2. The processing system according to claim 1, wherein the first axis of rotation is substantially vertical.

3. The processing system according to claim 1, wherein the loading area includes at least one upper insertion opening configured to enable a simultaneous positioning of a plurality of suction bags in receiving sites of the rotary support located opposite the at least one upper insertion opening.

4. The processing system according to claim 1, wherein the receiving sites are angularly offset relative to one another with respect to the first axis of rotation.

5. The processing system according to claim 1, which further includes a drive mechanism configured to drive the rotary support in rotation about the first axis of rotation.

6. The processing system according to claim 1, wherein the rotary support includes a plurality of holding members, each holding member being associated with a respective receiving site and being configured to hold in position a suction bag received in the respective receiving site when cutting said suction bag by the cutting element.

7. The processing system according to claim 6, each holding member includes at least one passage opening which is substantially vertical and which is configured to enable the passage of the cutting element.

8. The processing system according to claim 1, which further comprises at least one liquid wastes processing device, said liquid wastes processing device being disposed in the processing area and configured to collect and process liquid wastes originating from suction bags cut in the cutting area.

9. The processing system according to claim 1, which further comprises a storage device disposed in the storage area and configured to receive and store one or several processed suction bag(s), said storage device being at least partially open upwards.

10. The processing system according to claim 9, wherein the housing and the rotary support delimit a discharge opening located substantially vertically over the storage area, the discharge opening being configured to enable the fall by gravity of a suction bag processed beforehand, in the storage device when the respective receiving site is located substantially vertically over the storage area.

11. The processing system according to claim 1, which further comprises a cover movably mounted on an upper portion of the housing between an opening position in which the cover enables access to the rotary support and a closure position in which the cover prevents access to the rotary support.

12. The processing system according to claim 1, wherein each of the receiving sites includes a gripping device configured to alternately occupy a holding position in which the gripping device holds in position the suction bag positioned in one of the receiving sites, and a release position configured to release the suction bag.

13. The processing system according to claim 2, wherein the loading area includes at least one upper insertion opening configured to enable a simultaneous positioning of a plurality of suction bags in receiving sites of the rotary support located opposite the at least one upper insertion opening.

14. The processing system according to claim 13, wherein the receiving sites are angularly offset relative to one another with respect to the first axis of rotation.

15. The processing system according to claim 14, which further includes a drive mechanism configured to drive the rotary support in rotation about the first axis of rotation.

16. The processing system according to claim 15, wherein the rotary support includes a plurality of holding members, each holding member being associated with a respective receiving site and being configured to hold in position a suction bag received in the respective receiving site when cutting said suction bag by the cutting element.

17. The processing system according to claim 16, each holding member includes at least one passage opening which is substantially vertical and which is configured to enable the passage of the cutting element.

18. The processing system according to claim 17, which further comprises at least one liquid wastes processing device, said liquid wastes processing device being disposed in the processing area and configured to collect and process liquid wastes originating from suction bags cut in the cutting area.

19. The processing system according to claim 18, which further comprises a storage device disposed in the storage area and configured to receive and store one or several processed suction bag(s), said storage device being at least partially open upwards.

20. The processing system according to claim 19, wherein the housing and the rotary support delimit a discharge opening located substantially vertically over the storage area, the discharge opening being configured to enable the fall by gravity of a suction bag processed beforehand, in the storage device when the respective receiving site is located substantially vertically over the storage area.
